(19) United States
(12) Patent Application Publication
(10) Pub. No.: US 2026/0279565 A1
(43) Pub. Date: Sep. 17, 2026

(54) SYSTEM AND METHOD FOR SIX-AXIS FORCE SENSOR HAPTIC INTERACTION BASED ON THE INTERNET OF THINGS

(71) Applicant: CHENGDU QINCHUAN IOT TECHNOLOGY CO., LTD., Chengdu (CN)

(72) Inventors: Zehua SHAO, Chengdu (CN); Yong LI, Chengdu (CN); Jun WANG, Chengdu (CN); Chang SU, Chengdu (CN)

(73) Assignee: CHENGDU QINCHUAN IOT TECHNOLOGY CO., LTD., Chengdu (CN)

(21) Appl. No.: 19/678,112

(22) Filed: May 14, 2026

(30) Foreign Application Priority Data
Mar. 17, 2026 (CN) 202610341405.4

(51) Int. Cl. G16H 40/67; A61H 1/02; G16Y 10/60

(52) U.S. Cl. CPC G16H 40/67; A61H 1/02; G16Y 10/60; A61H 2201/5012; A61H 2201/5061; A61H 2201/5079

(57) ABSTRACT

A system, a method, and a storage medium for providing haptic interaction based on an IoT architecture and a six-axis force sensing assembly are provided. The method includes: collecting real-time force data of a target part of a patient, obtaining device operation data of an actuator; determining local control parameters based on rehabilitation data of the patient, the real-time force data, and the device operation data; in response to a packet loss rate of a communication link being less than or equal to a network tolerance threshold, obtaining a remote control instruction; correcting a driving parameter of the remote control instruction based on the real-time force data to determine a modified driving parameter, and driving the actuator to perform motion based on the modified driving parameter; and in response to the packet loss rate being greater than the network tolerance threshold, driving the actuator to perform motion based on a protective action parameter.

### CROSS-REFERENCE TO RELATED APPLICATIONS

This application claims priority to Chinese Patent Application No. 202610341405.4, filed on Mar. 17, 2026, the entire contents of which are hereby incorporated by reference.

### TECHNICAL FIELD

The present disclosure relates to the technical field of sensor control, and in particular to a system, a method, and a storage medium for six-axis force sensor haptic interaction based on an Internet of Things (IoT).

### BACKGROUND

In a remote rehabilitation scenario, a patient typically wears a rehabilitation exoskeleton or an end-effector traction-type rehabilitation device. Such devices integrate a six-axis force sensing assembly configured to sense, in real time, subtle muscle force variations, spasm torques, and active movement intentions of an affected limb during a rehabilitation process. By means of a medical Internet of Things (IoT) system, a rehabilitation device can upload haptic interaction information in real time to a cloud-based physician workstation or an artificial intelligence (AI) control center, thereby enabling remote evaluation, parameter adjustment, and rehabilitation process recording.

In remote rehabilitation control, due to network latency and jitter, the rehabilitation device is unable to respond in real time to actual haptic force changes of the patient, which may easily result in human-machine antagonistic interaction and even secondary injury. In addition, existing rehabilitation devices generally possess hardware capabilities such as traction force/torque adjustment, posture guidance, or compliant following. However, such hardware typically executes instructions locally in a simple manner and lacks an advance compensation mechanism based on muscle force trends, resulting in poor adaptability to remote collaboration and insufficient compliance.

Accordingly, it is desirable to provide a system, a method, and a storage medium for six-axis force sensor haptic interaction based on an Internet of Things (IoT), capable of performing compliant control compensation in advance before network data arrival, eliminating rigid impact sensations caused by transmission latency, and enabling precise evaluation in remote rehabilitation.

### SUMMARY

One or more embodiments of the present disclosure provide a system for six-axis force sensor haptic interaction based on an IoT, comprising: a management platform and a perception control platform. The perception control platform includes a rehabilitation device terminal and an actuator. The rehabilitation device terminal is configured to: collect, via a six-axis force sensing assembly and at a preset sampling frequency, real-time haptic force data of a target part of a patient; obtain device operation data of the actuator from the perception control platform; and transmit the real-time haptic force data and the device operation data to the management platform, wherein the device operation data includes a packet loss rate of a communication link; obtain local control parameters from the management platform, wherein the local control parameters include a network tolerance threshold and a protection action parameter; in response to the packet loss rate of the communication link being less than or equal to the network tolerance threshold, obtain a remote control instruction from the management platform, modify driving parameters of the remote control instruction based on the real-time haptic force data to determine modified driving parameters, and drive the actuator to perform motion based on the modified driving parameters; in response to the packet loss rate of the communication link being greater than the network tolerance threshold, drive the actuator to perform motion based on the protection action parameter. The management platform is configured to: determine the local control parameters based on rehabilitation data of the patient, the real-time haptic force data, and the device operation data, and transmit the local control parameters to the perception control platform.

One or more embodiments of the present disclosure provide a method for six-axis force sensor haptic interaction based on an IoT, implemented by a management platform and a rehabilitation device terminal of a perception control platform of a system for six-axis force sensor haptic interaction based on the IoT. The method comprises: collecting, via a six-axis force sensing assembly and at a preset sampling frequency, real-time haptic force data of a target part of a patient; obtaining device operation data of an actuator from the perception control platform; and transmitting the real-time haptic force data and the device operation data to the management platform, wherein the device operation data includes a packet loss rate of a communication link; determining local control parameters based on rehabilitation data of the patient, the real-time haptic force data, and the device operation data, and transmitting the local control parameters to the perception control platform, wherein the local control parameters include a network tolerance threshold and a protection action parameter; in response to the packet loss rate of the communication link being less than or equal to the network tolerance threshold, obtaining a remote control instruction from the management platform, modifying driving parameters of the remote control instruction based on the real-time haptic force data to determine modified driving parameters, and driving the actuator to perform motion based on the modified driving parameters; in response to the packet loss rate of the communication link being greater than the network tolerance threshold, driving the actuator to perform motion based on the protection action parameter.

One or more embodiments of the present disclosure provide a non-transitory computer-readable storage medium. The storage medium stores computer instructions that, when read by a computer, cause the computer to executes a method for psix-axis force sensor haptic interaction based on an IoT.

### BRIEF DESCRIPTION OF THE DRAWINGS

The present disclosure will be further elucidated by way of exemplary embodiments, which are described in detail with reference to the accompanying drawings. These embodiments are not limiting. In these embodiments, the same reference numerals denote the same structures, wherein:

FIG. 1 is a schematic platform diagram illustrating a system for six-axis force sensor haptic interaction based on an IoT according to some embodiments of the present disclosure.

FIG. 2 is a flowchart illustrating an exemplary process of a method for six-axis force sensor haptic interaction based on an IoT according to some embodiments of the present disclosure.

FIG. 3 is an exemplary flowchart illustrating determination of local control parameters according to some embodiments of the present disclosure.

FIG. 4 is an exemplary schematic diagram illustrating determination of a target driving torque according to some embodiments of the present disclosure.

FIG. 5 is an exemplary flowchart illustrating generation of a traction torque instruction and a zero-torque floating instruction according to some embodiments of the present disclosure.

### DETAILED DESCRIPTION

To more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose. The term “and/or”, as used herein, is merely a way of describing the associative relationship of an associated object, indicating that three relationships may exist, e.g., A and/or B, which may be represented as: A alone, both A and B, and B alone.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Furthermore, if the description of the embodiments in the present disclosure involves descriptions such as “first” and “second,” then these descriptions are for descriptive purposes only and are not to be construed as indicating or implying relative importance or implicitly specifying the quantity of the indicated technical features. Thus, features defined by “first” or “second” may explicitly or implicitly include at least one such feature.

Flowcharts are used in the present disclosure to illustrate the operations performed by the system according to some embodiments of the present disclosure. It should be understood that the operations described herein are not necessarily executed in a specific order. Instead, they may be executed in reverse order or simultaneously. Additionally, one or more other operations may be added to these processes, or one or more operations may be removed.

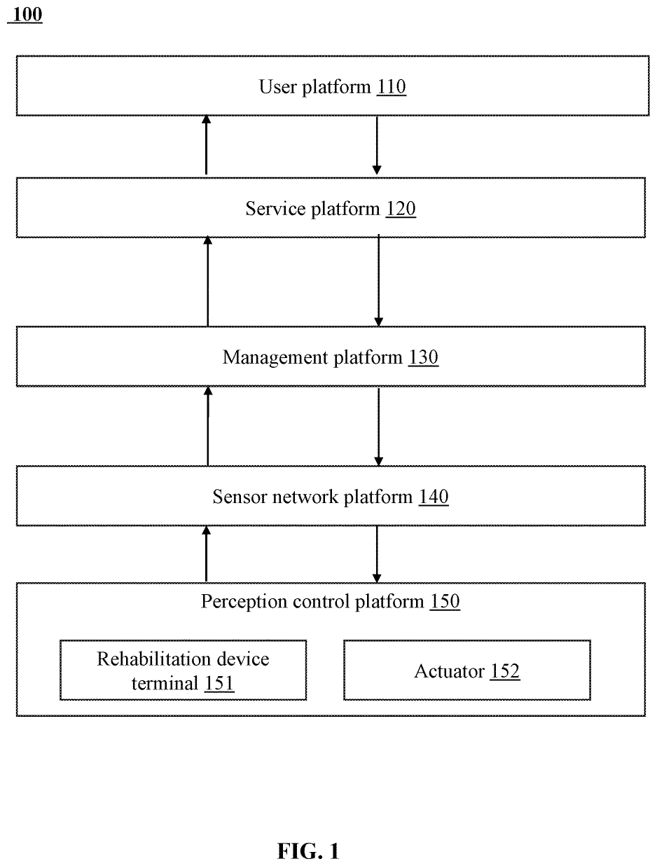

FIG. 1 is a schematic platform diagram of a system for six-axis force sensor haptic interaction based on an IoT according to some embodiments of the present disclosure.

In some embodiments, as shown in FIG. 1, a system 100 for six-axis force sensor haptic interaction based on an IoT (hereinafter referred to as “the system 100”) may comprise a management platform 130 and a perception control platform 150.

The management platform 130 refers to a platform used by a rehabilitation device company to supervise rehabilitation parameters. The rehabilitation parameters may include, but are not limited to, rehabilitation data of a patient, real-time haptic force data, device operation data, and local control parameters.

In some embodiments, the management platform 130 is configured to determine the local control parameters based on the rehabilitation data of the patient, the real-time haptic force data, and the device operation data, and transmit the local control parameters to the perception control platform 150.

In some embodiments, the management platform 130 may be implemented in a processor and/or a cloud server.

The perception control platform 150 refers to a platform that integrates a rehabilitation device and sensors and is configured to obtain sensing information and execute control information. The rehabilitation device refers to a device configured to perform rehabilitation treatment on a patient. In some embodiments, the rehabilitation device may comprise a rehabilitation exoskeleton, an end-effector traction-type rehabilitation device, or the like.

In some embodiments, the perception control platform 150 comprises a rehabilitation device terminal 151 and an actuator 152.

The rehabilitation device terminal 151 refers to a control terminal of the rehabilitation device and is configured to receive instructions and collect and process data.

The actuator 152 refers to a component of the rehabilitation device that directly generates movement and is configured to perform traction, rotation, and other actions according to driving parameters. In some embodiments, the actuator 152 may comprise an exoskeleton joint, an end effector (such as a mechanical gripper or a flexible clamp), a motor, a reduction gearbox, or the like.

In some embodiments, the rehabilitation device terminal 151 is configured to: collect, via the six-axis force sensing assembly and at a preset sampling frequency, real-time haptic force data of a target part of the patient; obtain device operation data of the actuator 152 of the perception control platform 150; and transmit the real-time haptic force data and the device operation data to the management platform 130, wherein the device operation data includes a packet loss rate of a communication link; obtain local control parameters from the management platform 130, wherein the local control parameters comprise a network tolerance threshold and a protection action parameter; in response to the packet loss rate of the communication link being less than or equal to the network tolerance threshold, obtain a remote control instruction from the management platform 130, modify driving parameters of the remote control instruction based on the real-time haptic force data to determine modified driving parameters, and drive the actuator 152 of the perception control platform 150 based on the modified driving parameters; and in response to the packet loss rate of the communication link being greater than the network tolerance threshold, drive the actuator 152 to perform motion based on the protection action parameter.

The six-axis force sensing assembly refers to a sensor capable of simultaneously measuring torques in six directions. The six directions may include three linear directions along an X-axis, a Y-axis, and a Z-axis, and three rotational directions about the X-axis, the Y-axis, and the Z-axis.

In some embodiments, the system 100 further comprises a user platform 110, a service platform 120, and a sensor network platform 140. The user platform 110, the service platform 120, the management platform 130, the sensor network platform 140, and the perception control platform 150 are sequentially communicatively connected.

The user platform 110 refers to an interactive platform oriented toward medical personnel and patients. In some embodiments, the user platform 110 may include at least one terminal device, such as a physician operation handle, a remote monitoring interface, a computer, a mobile phone, or the like.

The service platform 120 refers to a platform configured to transmit service requests. In some embodiments, the service platform 120 may be implemented as a server having communication capabilities and configured to perform data interaction with the user platform 110 and the management platform 130. For example, a patient may transmit a service request to the service platform 120 via the user platform 110, and the service platform 120 may report the service request and a level of urgency thereof to the management platform 130, so that a rehabilitation device company can perform response and decision-making. Medical personnel may upload medical examination reports, medical records, and other data via the user platform 110 and transmit the data to the management platform 130 through the service platform 120.

The sensor network platform 140 refers to a platform configured to perform sensing communication and to enable bidirectional data interaction between the management platform 130 and the perception control platform 150. For example, the sensor network platform 140 may include communication devices, servers, and various gateway devices.

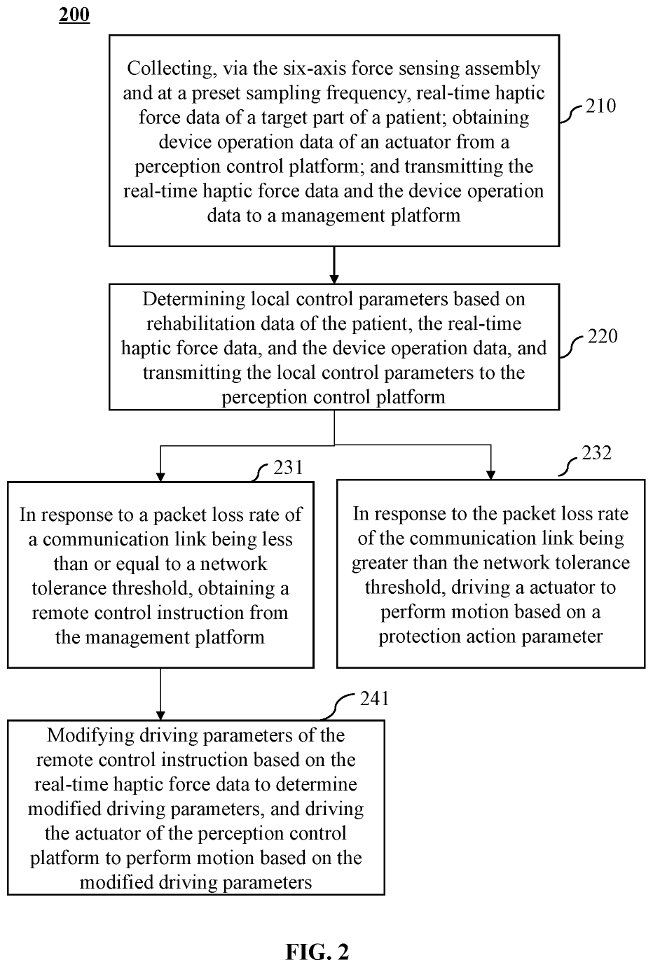

Further details regarding the above content may be found with reference to FIGS. 2-5 and the corresponding descriptions thereof.

In some embodiments of the present disclosure, by means of the system 100, an information operation closed loop can be formed among the various functional platforms. Under unified management of the management platform 130 and the perception control platform 150, coordinated and regulated operation can be achieved, thereby realizing informatization and intelligentization of haptic interaction based on the six-axis force sensing assembly.

FIG. 2 is a flowchart illustrating an exemplary process of a method for six-axis force sensor haptic interaction based on an IoT according to some embodiments of the present disclosure. As shown in FIG. 2, a process 200 comprises steps 210-241. Step 220 is performed by the management platform 130, and steps 210 and 230-241 are performed by the rehabilitation device terminal 151 of the perception control platform 150.

Step 210: collecting, via a six-axis force sensing assembly and at a preset sampling frequency, real-time haptic force data of a target part of a patient; obtaining device operation data of an actuator from the perception control platform; and transmitting the real-time haptic force data and the device operation data to the management platform.

For a description of the actuator, reference may be made to FIG. 1 and the corresponding description thereof.

The preset sampling frequency refers to a frequency at which the six-axis force sensing assembly collects the real-time haptic force data. In some embodiments, the preset sampling frequency may be preset based on experience.

The target part refers to a part requiring rehabilitation treatment. For example, the target part may be a diseased hand, the chest, or other body part of the patient.

The real-time haptic force data refers to data representing changes in muscle strength of the patient at a current moment. In some embodiments, the six-axis force sensing assembly may collect haptic signal data from a plurality of sampling points of the target part of the patient at the preset sampling frequency, and comprehensively extract, from the plurality of haptic signal data, data such as a muscle force applied to the target part, a torque, a directional force, a motion speed variation, a force mutation rate, and a direction reversal characteristic, which are used as the real-time haptic force data. The real-time haptic force data may be an average value of the plurality of haptic signal data.

The direction reversal characteristic refers to a characteristic reflecting a change in a force-exertion direction of the patient. In some embodiments, the direction reversal characteristic includes a count of changes in the force-exertion direction and a manner of change in the force-exertion direction. For example, the direction reversal characteristic may indicate that the force-exertion direction changes from “forward” to “backward,” or from “clockwise rotation” to “counterclockwise rotation.”

The device operation data refers to data reflecting an operating state of the actuator 152. In some embodiments, the device operation data includes a packet loss rate of a communication link.

The packet loss rate of the communication link refers to a packet loss rate of the communication link between the actuator 152 and the system 100.

In some embodiments, the device operation data may be obtained via the perception control platform 150. For example, the rehabilitation device terminal 151 may read the packet loss rate of the communication link via a network interface controller of the perception control platform 150.

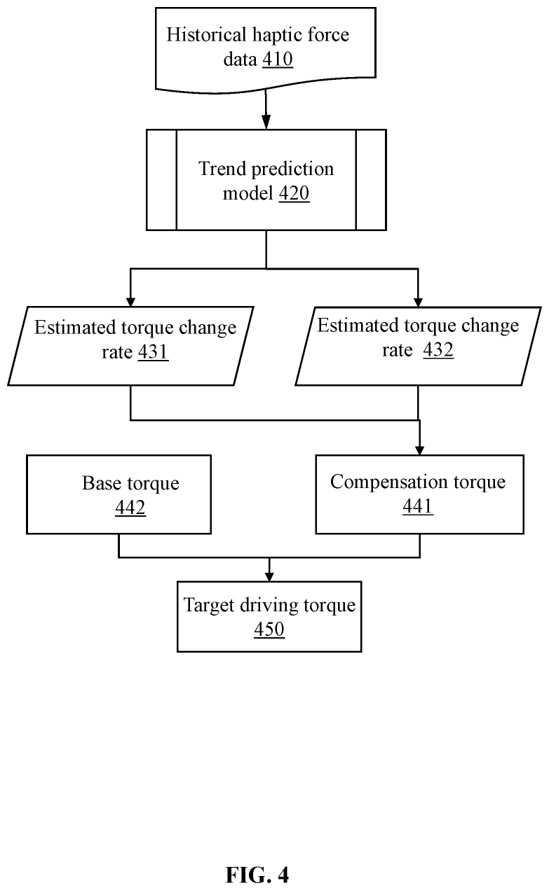

In some embodiments, the device operation data may further include a cumulative operating duration, a motor temperature, a count of compliant compensation operations performed by the rehabilitation device terminal 151, and a network latency value. Further details may be found with reference to FIG. 4 and the related description.

Step 220: determining local control parameters based on rehabilitation data of the patient, the real-time haptic force data, and the device operation data, and transmitting the local control parameters to the perception control platform.

The rehabilitation data refers to data reflecting a rehabilitation condition of the patient. In some embodiments, the rehabilitation data may include, but not limited to, a disease classification, a muscle strength grade, demographic information, a joint range of motion, and a muscle tone grade.

The disease classification refers to an exact medical diagnosis of the patient's condition, for example, stroke, spinal cord injury, Parkinson's disease, or postoperative orthopedic conditions.

The muscle strength grade refers to a quantitative assessment of muscle strength of the patient. In some embodiments, the muscle strength grade may be determined using a 0-5 grading scale according to a Lovett grading technique or a Kendall grading technique.

The demographic information includes age, gender, height, weight, or the like of the patient.

The joint range of motion refers to a maximum range through which each joint of the patient can actively or passively move.

The muscle tone grade refers to a quantitative assessment of muscle tension. In some embodiments, the muscle tone grade may be determined using a Modified Ashworth Scale.

In some embodiments, the rehabilitation device terminal 151 may obtain the rehabilitation data from medical examination reports, medical records, or the like, which may be acquired via the user platform 110.

The local control parameters refer to parameters used to determine a motion condition of the actuator 152 and to guide the actuator 152 to perform motion. In some embodiments, the local control parameters include a network tolerance threshold and a protection action parameter.

The network tolerance threshold refers to a threshold value used to determine whether a network condition is acceptable. In some embodiments, the network tolerance threshold may be determined based on requirements or experience.

The protection action parameter refers to one or more parameters associated with protective motion actions for the patient. The protective motion actions may include reducing at least one of a traction speed, a torque, or a traction force of the actuator 152 (such as an exoskeleton joint and/or an end effector). The protection action parameter may represent a control magnitude of the protective motion action (for example, a reduction magnitude).

In some embodiments, the management platform 130 may construct a first matching vector based on the rehabilitation data of the patient, the real-time haptic force data, and the device operation data, and retrieve a first vector database. A first vector labels corresponding to a first feature vector having a highest similarity to the first matching vector are used as the local control parameters. When a plurality of first feature vectors having the highest similarity exist, the local control parameters are determined based on an average value of the corresponding first vector labels.

In some embodiments, the first vector database includes multiple sets of first feature vectors and corresponding first vector labels. For example, the management platform 130 may screen historical rehabilitation data, historical haptic force data, and historical device operation data satisfying a preset condition from historical data as the first feature vectors, and use historical local control parameters corresponding to the first feature vectors as the corresponding first vector labels. The preset condition may include that a patient feedback effect is optimal or a rehabilitation outcome of the patient is optimal.

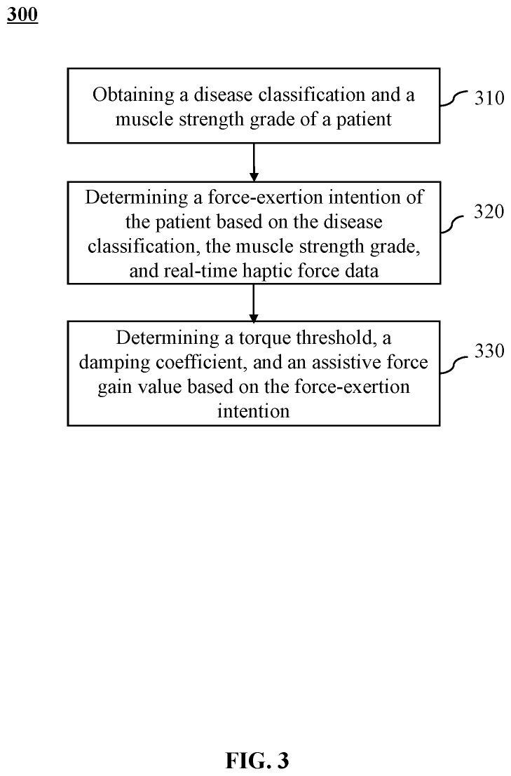

In some embodiments, the local control parameters further include a torque threshold, a damping coefficient, an assistive force gain value, and at least one early-warning trigger condition. Further details may be found with reference to FIGS. 3-5 and the related description.

Step 231: in response to the packet loss rate of the communication link being less than or equal to the network tolerance threshold, obtaining a remote control instruction from the management platform.

The remote control instruction refers to an instruction for controlling motion of the actuator 152. In some embodiments, the remote control instruction may include driving parameters.

The driving parameters refer to parameters for driving the actuator 152 to perform motion. In some embodiments, the driving parameters may include a torque, a traction force, and a traction speed of the actuator 152.

In some embodiments, in response to the packet loss rate of the communication link being less than or equal to the network tolerance threshold, the rehabilitation device terminal 151 obtains the torque, the traction force, and the traction speed of the actuator 152 from the management platform 130 as the remote control instruction.

Step 241: modifying the driving parameters of the remote control instruction based on the real-time haptic force data to determine modified driving parameters, and driving the actuator of the perception control platform to perform motion based on the modified driving parameters.

The modified driving parameters include a target torque, a target traction force, a target traction speed, or the like obtained by modifying the driving parameters.

In some embodiments, the management platform 130 may modify the driving parameters of the remote control instruction based on the real-time haptic force data through clustering analysis, so as to determine the modified driving parameters. For example, the management platform 130 may perform clustering processing on clustering vectors and a target vector using a clustering algorithm to determine a plurality of clusters, and designate a cluster to which the target vector belongs as a target cluster. An average value of labels corresponding to all clustering vectors in the target cluster is used as the modified driving parameters corresponding to the target vector. The clustering algorithm may include a K-Means clustering algorithm or the like.

In some embodiments, the target vector is constructed based on the real-time haptic force data and the remote control instruction. The clustering vectors may be determined based on historical data. For example, the management platform 130 may construct clustering vectors based on historical haptic force data and historical remote control instructions, and determine historical driving parameters of the actuator 152 corresponding to clustering vectors having highest subsequent feedback scores in the historical data as labels corresponding to the clustering vectors. The historical haptic force data refers to data representing changes in muscle strength of the patient at historical moments. The feedback score refers to a score provided by a user regarding a usage experience of the rehabilitation device. The feedback score may be obtained via the user platform 110.

In some embodiments, the rehabilitation device terminal 151 may drive the actuator 152 to perform motion based on the driving parameters. For example, the rehabilitation device terminal 151 may control a motor to drive an exoskeleton joint to rotate according to the target torque. As another example, the rehabilitation device terminal 151 may control a motor to drive an end effector to perform traction on the patient according to the target traction force and the target traction speed.

Step 232: in response to the packet loss rate of the communication link being greater than the network tolerance threshold, driving the actuator to perform motion based on the protection action parameter.

In some embodiments, in response to the packet loss rate of the communication link being greater than the network tolerance threshold, the rehabilitation device terminal 151 reduces the torque value, the traction force, and the traction speed of the actuator 152 according to a reduction magnitude specified in the protection action parameter, and drives the actuator 152 to operate according to the reduced driving parameters.

In some embodiments of the present disclosure, by obtaining the packet loss rate of the communication link and switching in real time between the remote control instruction and the protection action parameter to drive the actuator 152 to perform motion, interactive precision under normal communication conditions and local safety protection under abnormal network conditions are ensured.

FIG. 3 is an exemplary flowchart illustrating determination of local control parameters according to some embodiments of the present disclosure. As shown in FIG. 3, a process 300 is an exemplary process for determining local control parameters and comprises steps 310-330. In some embodiments, the process 300 is performed by the management platform 130.

In some embodiments, the local control parameters may further include a torque threshold, a damping coefficient, and an assistive force gain value.

The torque threshold refers to a maximum torque permitted to be output by the actuator during operation. For example, for a rehabilitation patient suffering from osteoporosis, the torque threshold may be set to a relatively low value (such as 10 N·m).

The damping coefficient is a parameter reflecting resistance characteristics exhibited by the actuator during motion and is used to simulate different motion environments or provide rehabilitation resistance. For example, in order to simulate resistance experienced during underwater rehabilitation training, a medium-level damping coefficient may be set.

The assistive force gain value is a coefficient for amplifying an output torque of the actuator 152. For example, when the assistive force gain value is 2, the actuator 152 outputs a torque equivalent to twice a torque currently exerted by the patient.

For further details regarding acquisition of the local control parameters, reference may be made to FIG. 2 and the related description thereof.

Step 310: obtaining a disease classification and a muscle strength grade of the patient.

For descriptions of the muscle strength grade and the disease classification and manners for acquiring the same, reference may be made to FIG. 2 and the related description thereof.

Step 320: determining a force-exertion intention of the patient based on the disease classification, the muscle strength grade, and real-time haptic force data.

For further details regarding the real-time haptic force data, reference may be made to FIG. 2 and the related description thereof.

The force-exertion intention refers to an action tendency or intention that the patient intends to perform at a current moment. In some embodiments, the force-exertion intention at least includes active force exertion and spasm.

In some embodiments, the management platform 130 may extract force-exertion features from the real-time haptic force data; and determine the force-exertion intention by querying a preset rule table based on the disease classification, the muscle strength grade, and the force-exertion features. The force-exertion features may include a force mutation rate, an initial speed, a frequency of a force signal, and directional consistency. The directional consistency refers to a stability degree of a direction of force and may be represented by a value between 0 and 1. In some embodiments, the directional consistency is inversely proportional to a variance of a vector direction of force exerted by the patient within a unit time period (for example, within 1 second).

The preset rule table is a correspondence table preset based on experience, and stores mapping relationships among force-exertion features, disease classifications, muscle strength grades, and force-exertion intentions. The mapping relationships may be set based on experience.

For example, the management platform 130 may determine the force-exertion intention as spasm in response to at least one of the force-exertion features, the disease classification, or the muscle strength grade satisfying a preset condition; otherwise, the force-exertion intention is determined as active force exertion. The preset condition may include that the force-exertion features satisfy a preset force condition, the disease classification corresponds to a preset disease (such as a disease prone to causing spasm), or the muscle strength grade is less than a muscle strength grade threshold. The preset force condition may include that the force mutation rate is greater than a mutation rate threshold, the initial speed is greater than a speed threshold, the frequency of the force signal is greater than a frequency threshold, and the directional consistency is less than a preset consistency threshold. The above thresholds may be preset based on experience.

Step 330: determining the torque threshold, the damping coefficient, and the assistive force gain value based on the force-exertion intention.

In some embodiments, the management platform 130 may determine the torque threshold, the damping coefficient, and the assistive force gain value based on the force-exertion intention in various manners. For example, if the force-exertion intention is active force exertion, the assistive force gain value of the actuator 152 may be increased by a first preset proportion. As another example, if the force-exertion intention is spasm, the torque threshold of the actuator 152 may be decreased by a second preset proportion, and the damping coefficient of the actuator 152 may be decreased by a third preset proportion. The first preset proportion, the second preset proportion, and the third preset proportion may be preset based on experience.

In some embodiments of the present disclosure, by comprehensively determining the force-exertion intention through a combination of the disease classification, the muscle strength grade, and the real-time haptic force data of the patient, and correspondingly adjusting the torque threshold, the damping coefficient, and the assistive force gain value, highly personalized rehabilitation control can be achieved. This approach not only avoids inadaptability of a single control parameter to patients with different medical conditions, but also accurately distinguishes between a patient's active motion intention and pathological abnormalities (such as spasm), thereby significantly improving pertinence and effectiveness of rehabilitation training while ensuring safety thereof.

FIG. 4 is an exemplary schematic diagram illustrating determination of a target driving torque according to some embodiments of the present disclosure.

In some embodiments, as shown in FIG. 4, the remote control instruction includes a base torque 442 and a compensation torque 441. The rehabilitation device terminal 151 may be further configured to: determine, based on a plurality of historical haptic force data 410 within a preset historical time period and through a trend prediction model 420, an estimated torque change rate 431 and an estimated motion deviation 432 for a future preset time period, wherein the trend prediction model 420 is a machine learning model; determine the compensation torque 441 based on the estimated torque change rate 431 and the estimated motion deviation 432; and determine a target driving torque 450 based on the base torque 442 and the compensation torque 441.

The base torque 442 refers to a theoretical torque required to drive the actuator under an ideal condition. For example, the base torque 442 may be preset based on experience.

The compensation torque 441 refers to an additional torque applied to modify the base torque in order to respond to the force-exertion intention of the patient.

The preset historical time period refers to a time period prior to a current moment. For example, the preset historical time period may be within 1 second, 3 seconds, or another duration prior to the current moment. The historical haptic force data 410 refers to data representing changes in muscle strength of the patient at historical moments. The rehabilitation device terminal 151 may collect historical haptic force data 410 at multiple moments within the preset historical time period via a six-axis force sensing assembly.

The estimated torque change rate 431 refers to a predicted rate of change of torque applied to the patient within a future preset time period. The future preset time period refers to a time period subsequent to the current moment. For example, the future preset time period may be within 10 minutes, 15 minutes, or another duration after the current moment.

The estimated motion deviation 432 refers to a predicted deviation between a motion state of the patient within the future time period and a planned motion state. The planned motion state includes a motion direction and a position planned by the rehabilitation device. The rehabilitation device terminal 151 may obtain the planned motion state uploaded by a user via the user platform 110. In some embodiments, the estimated motion deviation 432 includes a predicted position deviation value and a predicted direction deviation value. The predicted position deviation value refers to a deviation between an actual position of the user and a position in the planned motion state. The predicted direction deviation value refers to a deviation between an actual motion direction of the user and a motion direction in the planned motion state.

The trend prediction model 420 refers to a model configured to determine the estimated torque change rate and the estimated motion deviation for the future preset time period. In some embodiments, the trend prediction model 420 may include one or more of a Long Short-Term Memory (LSTM) model, a Gated Recurrent Unit (GRU) model, or a Transformer model.

In some embodiments, an input to the trend prediction model 420 may include the historical haptic force data 410 within the historical time period; and an output of the trend prediction model 420 may include the estimated torque change rate 431 and the estimated motion deviation 432 for the future preset time period.

In some embodiments, the trend prediction model 420 may be obtained through training using a plurality of labeled training samples. The training samples and corresponding labels may be obtained from historical data. In some embodiments, the management platform 130 may read a plurality of historical haptic force data collected within a first time period from the historical data as training samples; and obtain, from the historical data corresponding to the training samples, a plurality of actual torque values, motion directions, and motion positions within a second time period. An average torque change rate within the second time period and deviations of the motion direction and motion position from the planned motion state are used as labels corresponding to the training samples. A duration of the first time period is the same as a duration of the preset historical time period, and a duration of the second time period is the same as a duration of the future preset time period, wherein the first time period precedes the second time period.

In some embodiments, the management platform 130 may input the plurality of labeled training samples into an initial trend prediction model, construct a loss function based on the labels and output results of the initial trend prediction model, and iteratively update parameters of the initial trend prediction model using gradient descent or other techniques based on the loss function. When a preset condition is satisfied, model training is completed to obtain a trained trend prediction model. The preset condition may include convergence of the loss function or that a count of iterations reaches a threshold. After training is completed, the management platform 130 may deploy the trained trend prediction model to the rehabilitation device terminal 151.

In some embodiments, the rehabilitation device terminal 151 may construct a second matching vector based on the estimated torque change rate and the estimated motion deviation, retrieve a second vector database, and determine a second vector label corresponding to a second feature vector having a highest similarity to the second matching vector as the compensation torque. If a plurality of second feature vectors having the highest similarity exist, the compensation torque is determined as an average value of the plurality of corresponding second vector labels.

The second vector database includes a plurality of second feature vectors and corresponding second vector labels. In some embodiments, the rehabilitation device terminal 151 may construct the plurality of second feature vectors based on a plurality of historical torque change rates and a plurality of historical motion deviations. A second vector label corresponding to each second feature vector may be a historical compensation torque that achieved an optimal compensation effect in a historical compensation scenario corresponding to the second feature vector. The optimal compensation effect refer to a scenario in which a subsequent count of spasms experienced by the patient is minimized or a spasm duration is shortest.

In some embodiments, the rehabilitation device terminal 151 may be configured to: generate a negative compensation torque in response to the estimated torque change rate being greater than a preset spasm threshold; and generate a positive compensation torque in response to an actual movement direction of the patient being consistent with an active movement direction and a torque magnitude increasing.

The preset spasm threshold refers to a threshold value used to determine whether the patient is experiencing or is about to experience a spasm. The preset spasm threshold may be set based on experience.

The negative compensation torque refers to a damping or counteracting torque used to counteract a spasm. In some embodiments, in response to the estimated torque change rate being greater than the preset spasm threshold, the rehabilitation device terminal 151 may determine a direction of the compensation torque obtained from the second vector database to be opposite to the active motion direction, so as to obtain the negative compensation torque, until the estimated torque change rate is less than or equal to the preset spasm threshold. The manner for determining the compensation torque may refer to the foregoing description.

The actual motion direction of the patient may be obtained via the six-axis force sensing assembly.

The active motion direction refers to a preset active motion direction of the patient. In some embodiments, the rehabilitation device terminal 151 may use a motion direction in the planned motion state as the active motion direction of the patient.

In some embodiments, the rehabilitation device terminal 151 may calculate a vector angle between the actual motion direction and the active motion direction of the patient, and determine that the actual movement direction is consistent with the active motion direction in response to the vector angle being less than a preset angle threshold. The preset angle threshold may be set based on experience.

The torque magnitude refers to a magnitude of torque parsed from the real-time haptic force data. In some embodiments, the rehabilitation device terminal may obtain a plurality of historical torque values within a preset historical time period, and determine that the torque magnitude is increasing in response to the torque exhibiting an overall upward trend over time.

The positive compensation torque refers to a torque used to assist the patient in exerting force. In some embodiments, in response to the actual movement direction of the patient being consistent with being consistent with the active motion direction and the torque magnitude increasing, the rehabilitation device terminal 151 may determine a direction of the compensation torque retrieved from the second vector database to be the same as the active motion direction, so as to obtain the positive compensation torque.

In some embodiments of the present disclosure, by distinguishing between two typical haptic interaction scenarios, refined bidirectional regulation is achieved. On one hand, when the estimated torque change rate is greater than the preset spasm threshold (indicating a spasm risk), a negative damping torque is applied to ensure safety. On the other hand, when an active strengthening intention of the patient is identified, a positive compensation torque with increased assistive gain is generated to offset mechanical delay. This mechanism not only ensures safety of rehabilitation training, but also significantly improves real-time performance and smoothness of human-machine interaction by compensating for system latency.

The target driving torque refers to a torque ultimately transmitted to the actuator of the perception control platform. In some embodiments, the rehabilitation device terminal 151 may determine a vector sum of the base torque and the compensation torque as the target driving torque.

In some embodiments, the device operation data further includes a cumulative operating duration and a motor temperature. The rehabilitation device terminal 151 may be further configured to: calculate a health evaluation value of the rehabilitation device based on the cumulative operating duration and the motor temperature; determine an attenuation coefficient based on the health evaluation value; and determine the target driving torque based on the base torque, the compensation torque, and the attenuation coefficient.

The cumulative operating duration refers to a total duration during which the rehabilitation device has actually operated since leaving a factory or since a last comprehensive maintenance.

The motor temperature refers to a real-time temperature of a motor in the actuator 152.

In some embodiments, the rehabilitation device terminal 151 may obtain the cumulative operating duration and the motor temperature through sensors (such as a timer, a temperature sensor, or the like) within the rehabilitation device.

The health evaluation value refers to a quantitative score reflecting a health condition of the rehabilitation device. The lower the health evaluation value is, the poorer the health condition of the rehabilitation device.

In some embodiments, the health evaluation value is negatively correlated with the cumulative operating duration and negatively correlated with a difference between the motor temperature and a healthy temperature. The healthy temperature refers to a temperature of the motor during normal operation and may be set based on experience. In some embodiments, the rehabilitation device terminal 151 may perform normalization processing on the cumulative operating duration and on the difference between the motor temperature and the healthy temperature, perform weighted summation thereon, and determine the health evaluation value based on a weighted result. Weight coefficients are negative values and may be set based on experience. The normalization processing may include Min-Max normalization, Z-Score normalization, or the like.

In some embodiments, the device operation data further includes a count of compliant compensation operations of the rehabilitation device terminal, and the rehabilitation device terminal 151 is further configured to calculate the health evaluation value based on the count of compliant compensation operations, the cumulative operating duration, and the motor temperature.

The count of compliant compensation operations refers to a total count of times the actuator 152 triggers a compliant control action during a current rehabilitation training session of the patient. In some embodiments, the count of compliant compensation operations may be obtained via an internal controller or a sensor of the actuator 152.

Further details regarding the compliant control action may be found in the subsequent description.

In some embodiments, the health evaluation value may be negatively correlated with the count of compliant compensation operations. In some embodiments, the rehabilitation device terminal 151 may normalize the count of compliant compensation operations, and perform weighted summation on the normalized count of compliant compensation operations, the cumulative operating duration, and the difference between the motor temperature and the healthy temperature to obtain the health evaluation value. The weight coefficients may be preset based on experience and are negative values.

In some embodiments of the present disclosure, by introducing the count of compliant compensation operations as an indicator, a “dynamic load history” borne by the rehabilitation device can be incorporated into a health evaluation system. This enables more sensitive detection of mechanical fatigue caused by complex operating conditions such as high-frequency interaction and frequent anti-spasm responses, thereby improving the accuracy of the health evaluation value and preventing sudden equipment failures resulting from overlooked mechanical fatigue.

The attenuation coefficient is a proportional factor used to reduce the target driving torque and is generally a value less than 1. In some embodiments, the rehabilitation device terminal 151 may determine the attenuation coefficient based on the health evaluation value and a health standard value. The health standard value refers to a minimum health evaluation value at which the rehabilitation device is considered to be in a healthy state. The health standard value may be set based on experience.

In some embodiments, the rehabilitation device terminal 151 may determine the attenuation coefficient based on a first preset formula. For example, the first preset formula may be:

$$λ = \frac{\vert H - S \vert}{s} ,$$

where λ represents the attenuation coefficient, H represents the health evaluation value, and S represents the health standard value.

In some embodiments, the target driving torque is positively correlated with the base torque, the compensation torque, and the attenuation coefficient. For example, the rehabilitation device terminal 151 may obtain the target driving torque based on a second preset formula. The second preset formula may be:

$${T}_{target} = ( {T}_{base} + {T}_{comp} ) \times λ ,$$

where T_{target} represents the target driving torque, T_{base} represents the base torque, T_{comp} represents the compensation torque, and λ represents the attenuation coefficient.

In some embodiments of the present disclosure, by dynamically adjusting the attenuation coefficient based on the health evaluation value of the rehabilitation device, a “self-protection mechanism” of the rehabilitation device is achieved. When the rehabilitation device experiences long-term operational aging or sudden overheating, the output torque (i.e., the target driving torque) is automatically reduced. This not only effectively prevents device damage caused by overload and extends the service life of the rehabilitation device, but also avoids potential safety risks to the patient due to loss of control caused by equipment failure, thereby ensuring device-level safety during the rehabilitation training process.

In some embodiments, after determining the target driving torque, the rehabilitation device terminal 151 may convert the target driving torque into an underlying motor control signal, and transmit the motor control signal to the motor, such that the motor drives the actuator (such as a robotic arm or an exoskeleton joint) based on the target driving torque to move an affected limb.

In some embodiments, the local control parameters further include at least one early-warning trigger condition, and the real-time haptic force data includes a rate of change of velocity, a rate of abrupt torque change, and a direction reversal characteristic of the patient. The rehabilitation device terminal 151 is further configured to, in response to the rate of change of velocity, the rate of abrupt torque change, and the direction reversal characteristic satisfying the early-warning trigger condition, control the actuator to perform a compliant control action.

The rate of change of velocity refers to a rate of change, per unit time, of a movement velocity of a target body part of the patient.

The rate of abrupt torque change refers to an amplitude of change, per unit time, of a torque applied to the target body part of the patient.

In some embodiments, the rehabilitation device terminal 151 may obtain the rate of change of velocity and the rate of abrupt torque change through the six-axis force sensing assembly.

For more details regarding the direction reversal characteristic, reference may be made to FIG. 2 and the related description thereof.

The early-warning trigger condition refers to a condition for triggering the compliant control action. In some embodiments, the early-warning trigger condition may include that the rate of change of velocity is greater than a velocity change rate threshold, the rate of abrupt torque change is greater than a torque abrupt change rate threshold, and a direction reversal rate is greater than a direction reversal rate threshold. The direction reversal rate refers to a count of changes in a force exertion direction of the patient within a unit time (e.g., 1 second). The direction reversal rate may be a ratio of the count of changes in the force exertion direction in the direction reversal characteristic to the unit time.

In some embodiments, the velocity change rate threshold, the torque abrupt change rate threshold, and the direction reversal rate threshold may be set based on experience.

The compliant control action is a control action that reduces a risk of injury to the patient by reducing an output of the actuator. The compliant control action includes reducing an output torque, decreasing a traction velocity, reducing a count of reversals per unit time, or the like.

In some embodiments, in response to the rate of abrupt torque change being greater than the torque abrupt change rate threshold, the rehabilitation device terminal 151 may control the actuator 152 to reduce the output torque until the rate of abrupt torque change is less than or equal to the torque abrupt change rate threshold. In response to the rate of change of velocity being greater than the velocity change rate threshold, the rehabilitation device terminal 151 may control the actuator 152 to decrease the traction velocity until the rate of change of velocity is less than or equal to the velocity change rate threshold. In response to the direction reversal rate being greater than the direction reversal rate threshold, the rehabilitation device terminal 151 may control the motor of the actuator 152 to reduce the count of reversals per unit time until the direction reversal rate is less than or equal to the direction reversal rate threshold.

In some embodiments of the present disclosure, by formulating “targeted” early-warning trigger conditions based on the historical haptic force data of the patient, personalized safety protection is achieved. This mechanism is capable of intervening in advance based on a deterioration trend of data (such as an increase in abrupt change rate) before a safety threshold (e.g., a maximum force limit) is reached, and performing a compliant control action, thereby “softening” the system before a dangerous event occurs. As a result, the safety of rehabilitation training and adaptability to individual patient differences are greatly improved.

In some embodiments of the present disclosure, a trend prediction model is employed to predict future motion and torque trends in advance, thereby addressing a control lag problem caused by network latency. Through a compensation torque determination mechanism based on a vector database, intelligent compliant compensation is achieved by utilizing historically optimal experience. By combining attenuation control based on the health evaluation value and a protection mechanism based on trend early-warning, a comprehensive interactive system is constructed that not only provides precise rehabilitation assistance (positive compensation), but also effectively prevents spasm-induced injury (negative compensation/compliant control), while further possessing device self-protection capability.

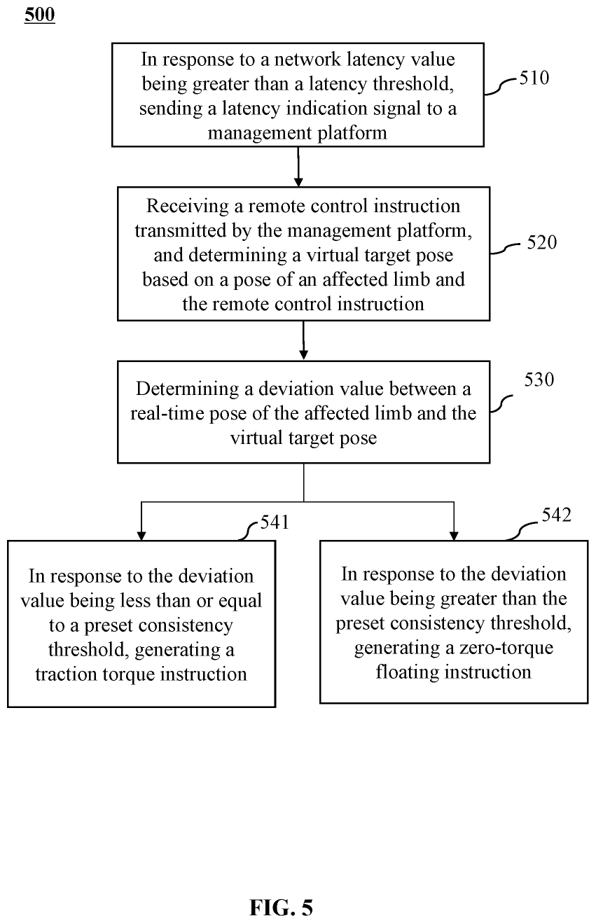

FIG. 5 is an exemplary flowchart illustrating generation of a traction torque instruction and a zero-torque floating instruction according to some embodiments of the present disclosure. As shown in FIG. 5, a process 500 includes steps 510 to 542, and the process 500 is executed by the rehabilitation device terminal 151 of the perception control platform 150.

In some embodiments, the device operation data further includes a network latency value.

The network latency value is a numerical value reflecting a delay condition of network transmission speed. In some embodiments, the rehabilitation device terminal 151 may send a Ping packet to the management platform 130, and determine a difference between a time at which the management platform 130 sends a response packet and a time at which the rehabilitation device terminal 151 receives the response packet as the network latency value.

Step 510: in response to the network latency value being greater than a latency threshold, sending a latency indication signal to the management platform.

In some embodiments, the latency threshold may be determined based on experience.

The latency indication signal is a signal indicating a network latency condition. In some embodiments, the latency indication signal may be predetermined based on experience. In some embodiments, the latency indication signal may trigger a user terminal (e.g., a physician handle) to generate a corresponding damping feedback force, or update a display status of the user terminal (e.g., a remote monitoring interface).

It is known that there are two scenarios: the network latency value being greater than the latency threshold, and the network latency value being less than or equal to the latency threshold. In some embodiments, in response to the network latency value being greater than the latency threshold, the rehabilitation device terminal 151 sends the latency indication signal to the management platform 130. For example, after receiving the latency indication signal, the management platform 130 may transmit the latency indication signal back to a handle at a physician side, and increase a damping coefficient of the physician handle by a preset magnitude, thereby making the operating handle “heavier.” The preset magnitude may be set based on experience.

In some embodiments, in response to the network latency value being less than or equal to the latency threshold, the rehabilitation device terminal 151 controls the actuator 152 to operate according to a target driving torque. For details regarding the target driving torque, reference may be made to the related description of FIG. 4.

Step 520: receiving a remote control instruction transmitted by the management platform, and determining a virtual target pose based on a pose of an affected limb and the remote control instruction.

The pose of the affected limb refers to a pose of a target body part of the patient. In some embodiments, the pose of the affected limb may include a position of the affected limb and an attitude of the affected limb.

The position of the affected limb refers to a position of the target body part of the patient. In some embodiments, the position of the affected limb may be represented by three-dimensional Cartesian coordinates (x, y, z) of a geometric center of the target body part.

The attitude of the affected limb refers to an orientation of the target body part of the patient. In some embodiments, the attitude of the affected limb may be represented by Euler angles. For example, the attitude of the affected limb may be represented as rotational amounts of the geometric center of the target body part about three principal axes (an X-axis, a Y-axis, and a Z-axis), respectively.

In some embodiments, the pose of the affected limb may be obtained by reading rotary encoders installed on exoskeleton joints, or by measuring a length and a direction of a traction cable of a terminal traction-type device.

The virtual target pose refers to a virtual, non-mandatory pose transmitted by a cloud server under high network latency. The virtual target pose includes a virtual target position and a virtual target attitude.

In some embodiments, the rehabilitation device terminal 151 may determine, based on real-time haptic force data, whether to follow the virtual target pose. A determination manner is described below.

In some embodiments, the rehabilitation device terminal 151 may receive the remote control instruction sent by the management platform 130, determine a linear velocity and an angular velocity based on the remote control instruction, and determine the virtual target pose based on the pose of the affected limb, the linear velocity, and the angular velocity.

In the remote control instruction, a traction velocity of the actuator 152 includes a linear velocity, and a torque includes an angular velocity. In some embodiments, the linear velocity includes linear velocities of the actuator 152 along the X-axis, the Y-axis, and the Z-axis. The angular velocity includes angular velocities of the actuator 152 about the X-axis, the Y-axis, and the Z-axis, and the angular velocity is an angular velocity associated with the base torque.

In some embodiments, the rehabilitation device terminal 151 may determine the virtual target pose based on the pose of the affected limb, the linear velocity, and the angular velocity through various manners. For example, the rehabilitation device terminal 151 may calculate the virtual target position based on the position of the affected limb and the linear velocity.

For example, coordinates of the virtual target position satisfy the following Formula (1):

$$x = {x}_{0} + {v}_{x} t \qquad ( 1 )$$

$$y = {y}_{0} + {v}_{y} t$$

$$z = {z}_{0} + {v}_{z} t ,$$

where the coordinates of the virtual target position are (x, y, z); coordinates of the position of the affected limb are (x₀, y₀, z₀); vₓ, v_{y}, and v_{z} are linear velocities of the actuator 152 along the X-axis, the Y-axis, and the Z-axis, respectively; and t is a preset time period, which may be determined based on experience.

As another example, the rehabilitation device terminal 151 may calculate the virtual target attitude based on the pose of the affected limb and the angular velocity. The virtual target attitude satisfies the following Formula (2):

$${R}_{x} = {r}_{x} + {ω}_{x} t \qquad ( 2 )$$

$${R}_{y} = {r}_{y} + {ω}_{y} t$$

$${R}_{z} = {r}_{z} + {ω}_{z} t ,$$

where Rₓ, R_{y}, and R_{z} represent rotational amounts of the geometric center of the target body part about the X-axis, the Y-axis, and the Z-axis in the virtual target attitude (i.e., the virtual target pose); rₓ, r_{y}, and r_{z} represent rotational amounts of the geometric center of the target body part about the X-axis, the Y-axis, and the Z-axis in a current attitude of the affected limb; and ωₓ, ω_{y}, and ω_{z} represent angular velocities of the actuator 152 about the X-axis, the Y-axis, and the Z-axis.

In some embodiments, when the network latency value is greater than the latency threshold, the remote control instruction is used to indicate the virtual target pose. The rehabilitation device terminal 151 may determine, based on real-time haptic force data, whether to follow the virtual target pose.

It is known that there are two scenarios: a torque applied to the target body part of the patient in the real-time haptic force data being less than a torque threshold, and the torque being greater than or equal to the torque threshold. For further details regarding the real-time haptic force data, reference may be made to FIG. 2. In some embodiments, in response to the torque applied to the target body part of the patient being less than the torque threshold, the rehabilitation device terminal 151 determines that the patient is not resisting and controls the actuator 152 to follow the virtual target pose. In response to the torque being greater than or equal to the torque threshold, the rehabilitation device terminal 151 determines that the patient is exerting resistance and controls the actuator 152 to remain in place.

In some embodiments of the present disclosure, by autonomously determining a following state of the virtual target based on real-time interaction forces, human-machine confrontation and forced displacement risks caused by remote instruction latency are avoided.

Step 530: determining a deviation value between a real-time pose of the affected limb and the virtual target pose.

In some embodiments, the rehabilitation device terminal 151 may calculate a deviation value between a three-dimensional position vector of the position of the affected limb and a three-dimensional position vector the virtual target position, and a deviation value between a three-dimensional rotation vector of the attitude of the affected limb and a three-dimensional rotation vector of the virtual target attitude; and determine the deviation value between the real-time pose of the affected limb and the virtual target pose based on the two calculated deviation values.

Step 541: in response to the deviation value being less than or equal to a preset consistency threshold, generating the traction torque instruction.

The preset consistency threshold may be determined based on experience.

The traction torque instruction refers to an instruction for controlling the actuator 152 to perform a traction action. In some embodiments, the traction torque instruction includes traction forces to be applied along an X direction, a Y direction, and a Z direction and rotational torques to be applied about three axes (the X-axis, the Y-axis, and the Z-axis).

In some embodiments, in response to the deviation value being less than or equal to the preset consistency threshold, the rehabilitation device terminal 151 may determine the traction torque instruction based on the deviation value between the three-dimensional vectors of the position of the affected limb and the virtual target position, and the deviation value between the three-dimensional rotation vectors of the attitude of the affected limb and the virtual target attitude, by using a preset algorithm. The preset algorithm may be a Proportional-Derivative (PD) control algorithm or the like.

Step 542: in response to the deviation value being greater than the preset consistency threshold, generating the zero-torque floating instruction.

The zero-torque floating instruction refers to an instruction for controlling the actuator 152 to unload force.

In some embodiments, in response to the deviation value being greater than the preset consistency threshold, the rehabilitation device terminal 151 may set a torque, a traction force, and a traction velocity of the actuator 152 to zero, so as to generate the zero-torque floating instruction.

In some embodiments of the present disclosure, by utilizing a virtual pose mapping and state feedback mechanism in a high-latency environment, an impact of communication jitter on control smoothness is reduced and cognitive deviation of a remote operator is eliminated.

In some embodiments, a non-transitory computer-readable storage medium is provided, wherein the storage medium stores computer instructions, and when a computer reads the computer instructions stored in the storage medium, the computer executes the above-described method for six-axis force sensor haptic interaction based on an IoT described in one or more embodiments of the present disclosure.

The basic concepts have been described above. Obviously, for those skilled in the art, the detailed disclosure above is merely illustrative and does not constitute a limitation on the present disclosure. Although not explicitly stated, those skilled in the art may make various modifications, improvements, and corrections to the present disclosure. Such modifications, improvements, and corrections are suggested within the present disclosure and thus remain within the spirit and scope of the exemplary embodiments of the present disclosure.

Meanwhile, the present disclosure uses specific terms to describe the embodiments herein. Terms such as “one embodiment,” “an embodiment,” and/or “some embodiments” mean that a certain feature, structure, or characteristic related to at least one embodiment of the present disclosure. Therefore, it is emphasized and noted that the terms “an embodiment,” “one embodiment,” or “an alternative embodiment” mentioned two or more times at different locations in the present disclosure do not necessarily refer to the same embodiment. Furthermore, certain features, structures, or characteristics in one or more embodiments of the present disclosure may be appropriately combined.

Furthermore, unless explicitly stated in the claims, the order of processing elements and sequences, the use of numbers or letters, or the use of other names in the present disclosure is not intended to limit the order of the processes and methods described herein. Although the foregoing disclosure discusses some currently considered useful inventive embodiments through various examples, it should be understood that such details are for illustrative purposes only, and the appended claims are not limited to the disclosed embodiments. On the contrary, the claims are intended to cover all modifications and equivalent combinations that conform to the essence and scope of the embodiments of the present disclosure. For example, although the system components described above may be implemented via hardware devices, they may also be realized solely through software solutions, such as installing the described system on existing servers or mobile devices.

Similarly, it should be noted that to simplify the presentation of the present disclosure and thereby aid in understanding one or more inventive embodiments, in the descriptions of the embodiments of the present disclosure above, various features may sometimes be grouped into a single embodiment, figure, or description thereof. However, this disclosure manner does not imply that the subject matter of the present disclosure requires more features than those mentioned in the claims. In fact, the features of an embodiment are fewer than all the features of a single embodiment disclosed above.

In some embodiments, numbers describing quantities or attributes are used. It should be understood that such numbers used in the description of the embodiments may, in some examples, be modified by the terms “approximately,” “about,” or “substantially.” Unless otherwise specified, “approximately,” “about,” or “substantially” indicates that the stated number allows for a variation of ±20%. Accordingly, in some embodiments, the numerical parameters used in the specification and claims are approximations, which may vary depending on the characteristics required by individual embodiments. In some embodiments, numerical parameters should consider the specified number of significant digits and adopt the method of general digit retention. Although the numerical ranges and parameters used to confirm their breadth in some embodiments of the present disclosure are approximations, in specific embodiments, such numerical values are set as accurately as possible within the feasible range.

For each patent, patent application, patent application publication, and other material, such as articles, books, specifications, publications, documents, etc., cited in the present disclosure, the entire content thereof is hereby incorporated by reference into the present disclosure. Excluded are application history documents that are inconsistent or conflict with the content of the present disclosure, and documents that limit the broadest scope of the claims of the present disclosure (whether currently or subsequently appended to the present disclosure). It should be noted that if the description, definition, and/or use of terms in the supplementary materials of the present disclosure are inconsistent or conflict with those in the present disclosure, the description, definition, and/or use of terms in the present disclosure shall prevail.

Finally, it should be understood that the embodiments described in the present disclosure are merely illustrative of the principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to those explicitly introduced and described herein.

What is claimed is:

1. A system for six-axis force sensor haptic interaction based on an Internet of Things (IoT), comprising: a management platform and a perception control platform, wherein the perception control platform includes a rehabilitation device terminal and an actuator, and the rehabilitation device terminal is configured to:
collect, via a six-axis force sensing assembly and at a preset sampling frequency, real-time haptic force data of a target part of a patient; obtain device operation data of the actuator from the perception control platform; and transmit the real-time haptic force data and the device operation data to the management platform, wherein the device operation data includes a packet loss rate of a communication link;
obtain local control parameters from the management platform, wherein the local control parameters include a network tolerance threshold and a protection action parameter;
in response to the packet loss rate of the communication link being less than or equal to the network tolerance threshold, obtain a remote control instruction from the management platform, modify driving parameters of the remote control instruction based on the real-time haptic force data to determine modified driving parameters, and drive the actuator to perform motion based on the modified driving parameters;
in response to the packet loss rate of the communication link being greater than the network tolerance threshold, drive the actuator to perform motion based on the protection action parameter;
wherein the management platform is configured to:
determine the local control parameters based on rehabilitation data of the patient, the real-time haptic force data, and the device operation data, and transmit the local control parameters to the perception control platform.

2. The system of claim 1, further comprising a user platform, a service platform, and a sensor network platform, wherein
the user platform, the service platform, the management platform, the sensor network platform, and the perception control platform are sequentially communicatively connected.

3. The system of claim 1, wherein the local control parameters further include a torque threshold, a damping coefficient, and an assistive force gain value, and
the management platform is further configured to:
obtain a disease classification and a muscle strength grade of the patient;
determine a force-exertion intention of the patient based on the disease classification, the muscle strength grade, and the real-time haptic force data; and
determine the torque threshold, the damping coefficient, and the assistive force gain value based on the force-exertion intention.

4. The system of claim 1, wherein the remote control instruction includes a base torque and a compensation torque, and the rehabilitation device terminal is further configured to:
determine, based on a plurality of historical haptic force data within a preset historical period and through a trend prediction model, an estimated torque change rate and an estimated motion deviation for a future preset period, wherein the trend prediction model is a machine learning model;
determine the compensation torque based on the estimated torque change rate and the estimated motion deviation; and
determine a target driving torque based on the base torque and the compensation torque, and control the actuator based on the target driving torque.

5. The system of claim 4, wherein the rehabilitation device terminal is further configured to:
in response to the estimated torque change rate being greater than a preset spasm threshold, generate a negative compensation torque; and
in response to an actual movement direction of the patient being consistent with an active movement direction and a torque magnitude increasing, generate a positive compensation torque.

6. The system of claim 4, wherein the local control parameters further include at least one early-warning trigger condition, the real-time haptic force data includes a rate of change of velocity, a rate of abrupt torque change, and a direction reversal characteristic of the target part of the patient, and the rehabilitation device terminal is further configured to:
in response to the rate of change of velocity, the rate of abrupt torque change, and the direction reversal characteristic satisfying the at least one early-warning trigger condition, control the actuator to perform a compliant control action.

7. The system of claim 4, wherein the device operation data further includes a cumulative operating duration and a motor temperature, and the rehabilitation device terminal is further configured to:
calculate a health evaluation value of a rehabilitation device based on the cumulative operating duration and the motor temperature;
determine an attenuation coefficient based on the health evaluation value; and
determine the target driving torque based on the base torque, the compensation torque, and the attenuation coefficient.

8. The system of claim 7, wherein the device operation data further includes a count of compliant compensation operations of the rehabilitation device terminal, and the management platform is further configured to:
calculate the health evaluation value based on the count of compliant compensation operations, the cumulative operating duration, and the motor temperature.

9. The system of claim 4, wherein the device operation data further includes a network latency value, and the rehabilitation device terminal is further configured to:
in response to the network latency value being greater than a latency threshold, transmit a latency indication signal to the management platform;
receive the remote control instruction transmitted by the management platform, determine a virtual target pose based on a pose of an affected limb and the remote control instruction;
determine a deviation value between a real-time pose of the affected limb and the virtual target pose;
in response to the deviation value being less than or equal to a preset consistency threshold, generate a traction torque instruction; and
in response to the deviation value being greater than the preset consistency threshold, generate a zero-torque floating instruction.

10. The system of claim 9, wherein, when the network latency value is greater than the latency threshold, the remote control instruction is used to indicate the virtual target pose, and the rehabilitation device terminal is further configured to:
determine, based on the real-time haptic force data, whether to follow the virtual target pose.

11. A method for six-axis force sensor haptic interaction based on an Internet of Things (IoT), implemented by a management platform and a rehabilitation device terminal of a perception control platform of a system for six-axis force sensor haptic interaction based on the IoT, the method comprising:
collecting, via a six-axis force sensing assembly and at a preset sampling frequency, real-time haptic force data of a target part of a patient; obtaining device operation data of an actuator from the perception control platform; and transmitting the real-time haptic force data and the device operation data to the management platform, wherein the device operation data includes a packet loss rate of a communication link;
determining local control parameters based on rehabilitation data of the patient, the real-time haptic force data, and the device operation data, and transmitting the local control parameters to the perception control platform, wherein the local control parameters include a network tolerance threshold and a protection action parameter;
in response to the packet loss rate of the communication link being less than or equal to the network tolerance threshold, obtaining a remote control instruction from the management platform, modifying driving parameters of the remote control instruction based on the real-time haptic force data to determine modified driving parameters, and driving the actuator to perform motion based on the modified driving parameters;
in response to the packet loss rate of the communication link being greater than the network tolerance threshold, driving the actuator to perform motion based on the protection action parameter.

12. The method of claim 11, wherein the local control parameters further include a torque threshold, a damping coefficient, and an assistive force gain value, and the method further comprises:
obtaining a disease classification and a muscle strength grade of the patient;
determining a force-exertion intention of the patient based on the disease classification, the muscle strength grade, and the real-time haptic force data; and
determining the torque threshold, the damping coefficient, and the assistive force gain value based on the force-exertion intention.

13. The method of claim 11, wherein the remote control instruction includes a base torque and a compensation torque, and the modifying driving parameters of the remote control instruction based on the real-time haptic force data to determine modified driving parameters, and driving the actuator to perform motion based on the modified driving parameters includes:
determining, based on a plurality of historical haptic force data within a preset historical period and through a trend prediction model, an estimated torque change rate and an estimated motion deviation for a future preset period, wherein the trend prediction model is a machine learning model;
determining the compensation torque based on the estimated torque change rate and the estimated motion deviation; and
determining a target driving torque based on the base torque and the compensation torque, and controlling the actuator based on the target driving torque.

14. The method of claim 13, wherein the determining the compensation torque based on the estimated torque change rate and the estimated motion deviation includes:
in response to the estimated torque change rate being greater than a preset spasm threshold, generating a negative compensation torque; and
in response to an actual movement direction of the patient being consistent with an active movement direction and a torque magnitude increasing, generating a positive compensation torque.

15. The method of claim 13, wherein the local control parameters further include at least one early-warning trigger condition, the real-time haptic force data includes a rate of change of velocity, a rate of abrupt torque change, and a direction reversal characteristic of the target part of the patient, and the method further comprises:
in response to the rate of change of velocity, the rate of abrupt torque change, and the direction reversal characteristic satisfying the at least one early-warning trigger condition, controlling the actuator to perform a compliant control action.

16. The method of claim 13, wherein the device operation data further includes a cumulative operating duration and a motor temperature, and the determining a target driving torque based on the base torque and the compensation torque includes:
calculating a health evaluation value of a rehabilitation device based on the cumulative operating duration and the motor temperature;
determining an attenuation coefficient based on the health evaluation value; and
determining the target driving torque based on the base torque, the compensation torque, and the attenuation coefficient.

17. The method of claim 16, wherein the device operation data further includes a count of compliant compensation operations of the rehabilitation device terminal, and the method further comprises:
calculating the health evaluation value based on the count of compliant compensation operations, the cumulative operating duration, and the motor temperature.

18. The method of claim 13, wherein the device operation data further includes a network latency value, and the method further comprises:
in response to the network latency value being greater than a latency threshold, transmitting a latency indication signal to the management platform;
receiving the remote control instruction transmitted by the management platform, determining a virtual target pose based on a pose of an affected limb and the remote control instruction;
determining a deviation value between a real-time pose of the affected limb and the virtual target pose;
in response to the deviation value being less than or equal to a preset consistency threshold, generating a traction torque instruction; and
in response to the deviation value being greater than the preset consistency threshold, generating a zero-torque floating instruction.

19. The method of claim 18, wherein, when the network latency value is greater than the latency threshold, the remote control instruction is used to indicate the virtual target pose, and the method further comprises:
determining, based on the real-time haptic force data, whether to follow the virtual target pose.

20. A non-transitory computer-readable storage medium storing computer instructions that, when read and executed by a computer, cause the computer to perform a method for six-axis force sensor haptic interaction based on an Internet of Things (IoT), the method comprising:
collecting, via a six-axis force sensing assembly and at a preset sampling frequency, real-time haptic force data of a target part of a patient; obtaining device operation data of an actuator from a perception control platform; and transmitting the real-time haptic force data and the device operation data to a management platform, wherein the device operation data includes a packet loss rate of a communication link;
determining local control parameters based on rehabilitation data of the patient, the real-time haptic force data, and the device operation data, and transmitting the local control parameters to the perception control platform, wherein the local control parameters include a network tolerance threshold and a protection action parameter;
in response to the packet loss rate of the communication link being less than or equal to the network tolerance threshold, obtaining a remote control instruction from the management platform, modifying driving parameters of the remote control instruction based on the real-time haptic force data to determine modified driving parameters, and driving the actuator to perform motion based on the modified driving parameters;
in response to the packet loss rate of the communication link being greater than the network tolerance threshold, driving the actuator to perform motion based on the protection action parameter.
